(19) United States
(12) Patent Application Publication
(10) Pub. No.: US 2008/0177319 A1
(43) Pub. Date: Jul. 24, 2008

(54) Expansion Rod, Self-Adjusting

(76) Inventor: Helmut Schwab, (US)

Correspondence Address: Helmut Schwab, 61 Westcott Road, Princeton, NJ, 08540, US

(21) Appl. No.: 11/608,816

(22) Filed: Dec. 9, 2006

(51) Int. Cl. A61B 17/58

(52) U.S. Cl. 606/257

(57) ABSTRACT

The design of a rod is disclosed that can serve, among other more general applications, as an implant to stimulate the reduction of spinal deformity. Different from common solid rod implants, the here-disclosed rod provides an almost uniform pressure between a chosen maximum and minimum by means of an internal spring and by expanding its length to adjust for progressing spinal growth or adjustment. The rod contains an internal ratchet which permits a piston to expand only outward, thereby providing easy installation and the expansion of the rod over time as the spine adjusts. The controlled expansion occurs in surgical applications through occasional body flexing, thereby avoiding additional interventions for adjustment.

### ABSTRACT/SUMMARY

A rod contains an internal ratchet which permits a piston to expand only outward, thereby providing an expansion of the rod. The expansion occurs, in the case of orthopedic applications, through body flexing. An internal spring and piston restricts the expansion within a pre-selected pressure range.

### CROSS REFERENCE TO RELATED APPLICATIONS, GENERAL BACKGROUND

Orthopedic deformations, specifically of the spine, call for corrective therapy. It is known that temporarily forced adjustment of form can lead not only to short term forced recovery of form but also to long-term natural and permanent recovery of form. Therefore, it is customary to implant various rods or other mechanical elements and attach those by means of screws or other fasteners to bone elements to provide such forced adjustment of form.

A specific problem arises with the natural adjustment or growth of bone or spine segments. Not only should an optimal amount of pressure of the inserted element against the bone be maintained, but, actually, an adjustment of length of the inserted element may become necessary.

The here-described invention “Expansion Rod, Self-Adjusting” provides the design of a rod which supplies expansion force (in contrast to other designs that provide pressure or contraction) and is self adjusting within a wide range of natural growth within the subject application.

A specific benefit of “self-adjustment” lies in the elimination of additional surgical intervention to provide for such adjustment as natural growth or incremental form adjustment occurs.

### SPECIFIC EXAMPLE OF AN APPLICATION

When curvature of the spine (scoliosis) occurs, quite often among still growing juveniles, a forced adjustment of the spine toward a more natural curvature is a common surgical therapy.

At present, solid rods attached to sequences of vertebrae by means of screws are the common solution.

As the spine would respond by reducing the curvature or as the juvenile spine grows, surgical adjustments of the rods would become necessary.

An expansion of the arch within the pathological curvature appears as a desirable alternative. However, this approach was not used in the past due to the problems with ongoing correction of such expansion in order to maintain expansion pressure as the spine responds or grows.

The proposed “Expansion Rod, Self-Adjusting” solves these problems and offers an ideal new tool to the orthopedic surgeon for scoliosis correction without ongoing adjustment requirements.

### SUMMARY OF THE INVENTION

### Objects:

It is the object of this invention to provide a simple device—for example, as an implant for orthopedic applications in scoliosis—to exert a uniform pressure within a given range independent of linear variations of the substrate to which this device may become attached—for example, growth of formation of the spine. This is intended, in case of a scoliosis application, to provide for a therapeutic change of the curvature of the spine.

### DESCRIPTION OF THE DRAWING OF THE STRUCTURE OF THE INVENTION “EXPANSION ROD, SELF-ADJUSTING”

FIG. 1 presents a schematic illustration of the invention. Actual executions would be more slim and elongated for suitability as implants—but more difficult to pictorially represent, especially in cross-sections.

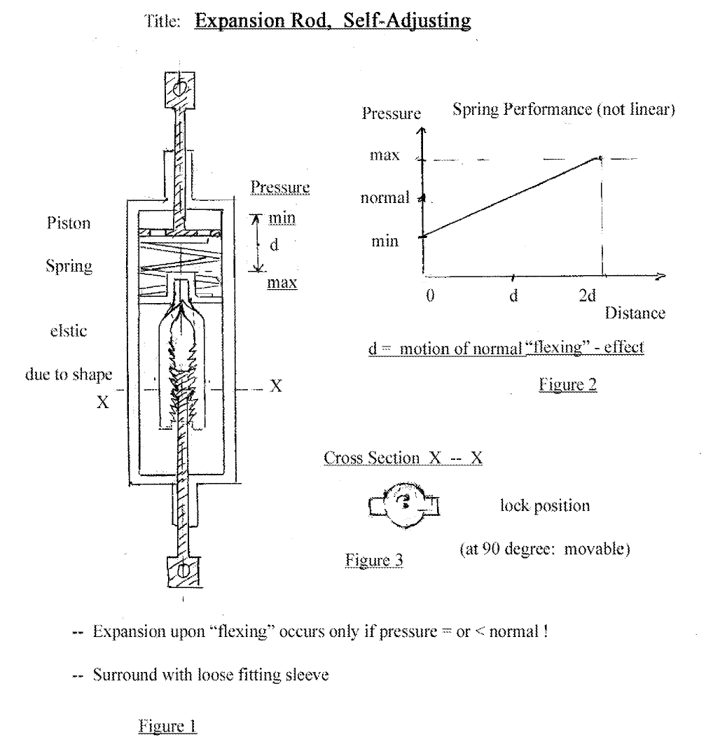

The “Rod” contains two segments, which appear on FIG. 1 as the upper segment and the lower segment. In practical applications, however, the rods may be implanted at any angle, horizontally, or, in reference to FIG. 1, upside down.

### Description of the Lower Section of the “Expansion Rod, Self-Adjusting”:

This section contains a ratchet system and a piston extending outward, out or the rod, to be permanently anchored by means of a screw or other fastener to a vertebrae or other bone structure, for example at the bottom within the inner arch of a curved section of the spine in a case of scoliosis—or within the inner section of a contorted section that requires rotation adjustment—mostly both.

The ratchet contains a fixed structure attached to the main body of the rod consisting of two blades with elastic support to permit the ratchet to function. Single blades with, for example, only a sequence of holes for ratchet functions could be used alternatively—or any other ratchet mechanism that would allow the movement of the piston outward, but not its being pushed into the rod chamber.

The piston is provided with a head featuring a short ratchet pattern on each side; see its cross section as shown within FIG. 3. This permits the movement of the piston outward, but not its being pushed into the rod chamber against the fixed ratchet pattern on the blades.

Rotation of the piston by 90 degrees, as upon or before implantation, liberates the piston from the ratchet engagement and allows the movement of the piston far into the rod—for the shortest possible configuration of the “Expansion Rod, Self-Adjusting”. Rotation back to the operating position, before anchoring it, engages the ratchet arrangement for proper operation.

### Description of the Upper Section of the “Expansion Rod, Self-Adjusting”

This section contains a spring that pushes against a piston which extends out of the rod. The end of the piston should be attached to that point of the spine or other vertebrae, which require the therapeutic extension for intended form or rotation correction.

The strength of the spring shall be designed such that, at its maximum possible free extension within the chamber, it supplies the minimally necessary pressure for the intended use. Furthermore, the spring and chamber shall be designed such that when the piston is moved as far into the chamber as mechanically possible, the maximally tolerable pressure is applied to the piston. This is indicated by the small graph showing the spring performance in FIG. 2.

In case of an application in scoliosis correction, it is known that the unrestricted spine permits a certain flexibility to permit body movement. This would translate into distance variations between the two points to which the “Expansion Rod, Self-Adjusting” would be anchored. This distance variation resulting from a normal degree of body flexing shall be designated as “d”, as shown in FIG. 1.

The space in the “Expansion Rod, Self-Adjusting” provided for piston movement in the “upper” section shall be equal to 2×d. Adjustment features for correction of this distance within the rod after manufacturing but before implantation can be easily added to the rod design.

### Detailed Description of the Functioning of the Invention “Expansion Rod, Self-Adjusting”:

Upon proper implantation of the “Expansion Rod, Self-Adjusting”, the rod should initially not exert any expansion pressure—in order to facilitate implantation—but should correspond in length to the uncorrected distance between its anchoring points.

In this case, the piston in the “upper” section will be pushed by the spring to its top position, stopped at this point by the rod chamber.

When properly installed, the ratchet system in the “lower” section of the “Expansion Rod, Self-Adjusting” will be in its shortest (most “pushed-in) or a very short position by way of adjustment as described before—just long enough to be anchored easily.

After implantation, upon a demanded flexing movement by the patient as prescribed by the physician, such that the distance in the rod area would increase, the piston will pull against the rod assembly and, thereby, will result in a corresponding movement in the ratchet section, resulting in a permanent lengthening of the “Expansion Rod, Self-Adjusting”.

Repeated flexing movements will quickly leave substantial pressure on the piston, which will push it against the spring.

If this pressure exceeds the installed and spring-provided “minimum” pressure, the piston will not return to its upper-most position within the rod assembly against the chamber after flexing.

Consequently, a following flexing motion would have to exceed the open space left between the piston and the chamber before any additional ratchet adjustment or lengthening of the rod takes place. As a matter of fact, when the pressure which the rod exerts on its anchoring points is about in the middle of the desired range and the spring compressed accordingly, any flexing movement will result in no further extension of the rod length.

This is the functioning which renders the “Expansion Rod, Self-Adjusting”, upon periodic flexing motions from time to time, to be self-adjusting to the mid-pressure length.

The same functioning occurs in rotation adjustment applications.

The remaining travel space of the spring provides for safety protection of the patient in case of a violent motion or accident—restricting the pressure of the rod to the area between middle and maximum of the spring design.

A patent is claimed for a mechanical rod which is resistant to compression but which expands when the pressure against its endpoints sinks below a given limit, then becoming compression resistant at the increased length, whereby the expansion pressure is predetermined, wherein the improvement comprises the following:

1. A rod with anchoring points at each end where one end is designed as reaching a piston within the rod such that, by means of a ratchet arrangement, the piston is allowed to move only in one direction, namely outward, resulting in an expansion of the total rod.

2. A rod according to claim 1 with a ratchet arrangement designed to be active only in a radial segment of the piston corresponding to claim 1, thereby permitting the disengagement of the ratchet function by simple rotation of the piston.

3. A rod according to claim 1 within which the unidirectional expansion is provided by other means than a ratchet, for example, by a screw-like thread that operates in one direction only.

4. A rod with a piston arrangement operating against a pressure provided within the design of the rod.

5. A rod according to claim 4 where the pressure provided within the design of the piston of the rod under claim 4 is to be provided by a spring, a compressed gas, or any other means of elastic motion resistance.

6. A rod according to claim 4 where the pressure provided within the design of the piston of the rod under claim 4 is to be selected such that maximum extension of the piston results in lowering the pressure to a pre-selected “minimum” pressure, extension to the midpoint results in the pressure to a pre-selected “standard” pressure, and extension to the minimum results in pressure to a pre-selected “maximum” pressure.

7. A rod according to claim 4 with a compression area corresponding to claim 4 to 6 where the distance for piston travel from “minimum” to “standard” pressure corresponds to a pre-selected externally provided motion amount as, for example, provided by standard body flexing in orthopedic applications.

8. A rod according to claim 4 with a compression area corresponding to claim 4 to 6 where the distance for piston travel from “standard” to “maximum” pressure corresponds to a pre-determined externally provided motion amount as, for example, occurring upon involuntary extreme body flexing in orthopedic applications, as when falling or in accidents.

9. A rod according to claim 4 where the compression area as claimed under claim 4 is not provided by a piston but by another compression design of the rod, for example, hollow rod segments sliding into each other filled with either a spring, compressed gas, or other elastic means.
